(19) United States
(12) Patent Application Publication
(10) Pub. No.: US 2022/0021964 A1
(43) Pub. Date: Jan. 20, 2022

(54) A Headphone with Motion Sensors

(71) Applicant: Innovation Sound Technology Co., LTD., Guangdong (CN)

(72) Inventors: Shunming YUEN, Guangdong (CN); Wai Yin MUNG, Guangdong (CN); Ka Ming WU, Guangdong (CN)

(21) Appl. No.: 16/611,143

(22) PCT Filed: Oct. 25, 2018
(86) PCT No.: PCT/CN2018/111842

§ 371 Date: Sep. 27, 2021

(30) Foreign Application Priority Data
Oct. 22, 2018 (CN) 201821711566.5

(51) Int. Cl. H04R 1/10; A61B 5/11; H04R 1/02; H04R 5/033

(52) U.S. Cl. CPC H04R 1/1041; A61B 5/1116; G06F 3/011; H04R 5/0335; H04R 1/028

(57) ABSTRACT

The utility model discloses a headphone with motion sensors, comprising a left channel earphone, a right channel earphone and a main electronic circuit. Motion sensors are provided in the main electronic circuit, the left channel earphone and the right channel earphone respectively. The said motion sensors are all linked to a microprocessor. The utility model provides three motion sensors in the headphone casing and the main electronic circuit to sense the neck position of a person who uses or wears the headphone, sending a prompt message after a long time of lowering head. It helps a user to take a rest in time, and relieve his/her neck pressure.

### FIELD OF THE INVENTION

The utility model relates to a headphone, and in particular to a headphone with motion sensors.

### BACKGROUND OF THE INVENTION

As mobile phones become more and more versatile, many people look down at their mobile phones longer and longer, which easily causes cervical dislocation. Studies indicate that when people look down at mobile phones with their necks bent at an angle of 50-60 degrees, the pressure acting on their cervical vertebrae weighs 30 Kg, which is equivalent to the weight of a 7-year-old child. Many young people often use their mobile phones with a head-lowering gesture, which pulls neck muscles, and will eventually reduce or even eliminate the curvature of cervical vertebrae, causing cervical dislocation, tilting cervical vertebrae forward, and leading to neck pain, headache, and even bone spurs. In severe cases, spinal nerves will be affected, and paralysis, even paralysis occurs.

That young people use mobile phones for a long time may cause “cell phone neck”, which means that the cervical vertebra has begun to show signs of rigidity. If the situation persists, cervical aging may occur earlier. If the sign tends to be severe, symptoms such as stiff neck and shoulders, headache, double chins, and restlessness may develop.

Content of the Utility Model

The utility model aims to provide a headphone with motion sensors to solve the problem brought up in the above background technology.

To achieve the above object, the present invention provides the following technical schemes:

A headphone with motion sensors, comprising a left channel earphone, a right channel earphone and a main electronic circuit, wherein the motion sensors are provided in the main electronic circuit, the left channel earphone and the right channel earphone, and are all linked to a microprocessor.

As a further scheme of the utility model, the said main electronic circuit is further provided with a microprocessor, a D/A conversion circuit, an amplifier and a Bluetooth module; a speaker is disposed in the left channel earphone and the right channel earphone respectively, and is also connected to the amplifier that is connected with the D/A conversion circuit; the D/A conversion circuit is linked to the microprocessor.

As a further scheme of the utility model, the said main electronic circuit is disposed within the headphone beam.

As a further scheme of the utility model, the said main electronic circuit is further provided with a vibrator that is connected to the microprocessor.

Compared with the prior art, the beneficial effects of the utility model are as follows:

The utility model provides three motion sensors in the headphone casing and the main electronic circuit to sense the neck position of a person who uses or wears the headphone, sending a prompt message after a long time of lowering head. It helps the user to take a rest in time, and relieve his/her neck pressure.

### BRIEF INTRODUCTION OF THE DRAWINGS

FIG. 1 is a structural schematic diagram of the utility model.

FIG. 2 is a schematic diagram showing the structure principle of the utility model.

### DETAILED EMBODIMENT

The technical scheme in the embodiment of the utility model will be clearly and completely described as follows with reference to the accompanying drawings. Apparently, the described embodiments are only a part of that of the invention, and not all of them. All other embodiments obtained by those skilled in the art based on the embodiments of the utility model without making creative efforts shall fall within the scope of protection of the present invention.

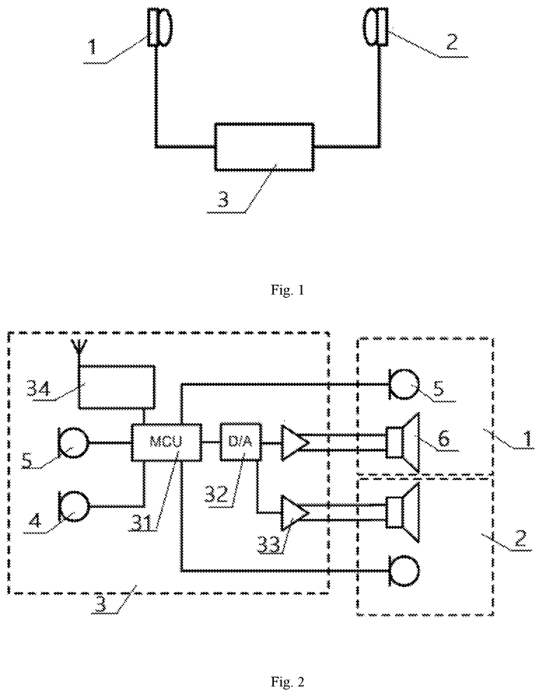

Refer to FIG. 1˜2 for a headphone with motion sensors, as proposed in the embodiment of the present invention, comprising a left channel earphone 1, a right channel earphone 2, and a main electronic circuit 3. The said main electronic circuit (3) is internally provided with a microprocessor (31), a D/A conversion circuit (32), an amplifier (33) and a Bluetooth module (34); the said amplifier 33 is respectively connected to the speakers 6 in the left channel earphone 1, the right channel earphone 2 and the D/A conversion circuit 32 that is linked to the microprocessor 31; the main electronic circuit 3, the left channel earphone 1, and the right channel earphone 2 are provided with motion sensors 5 linked to the microprocessor 31; the main electronic circuit 3 is disposed within the headphone beam. The motion sensors 5 in the left channel earphone 1 and right channel earphone 2 are used to sense head movement. When the head is lowered, the microprocessor 31 collects the data transmitted from the motion sensor 5; after the head remains in a lowered state for a long time, the microprocessor 31 gives an audible prompt through the headphone speaker 6, or transmits the information to the earphone connected to the mobile phone through the Bluetooth module 34, prompting the user who has used the mobile phone for a long time to take a rest properly. The motion sensor in the head beam of the headphone serves as a function of collecting neck movement data when the earphone is not worn, but hung on the neck.

A vibrator 4 is further disposed in the main electronic circuit 3, connected to the microprocessor 31, for giving a supplementary vibration prompt in addition to the audible prompt, thereby providing a better prompt effect.

For those skilled in the art, apparently the utility model is not limited to the details given in the above exemplary embodiments. The utility model can be embodied in other specific forms without departing from the spirit or essential characteristics of the utility model. Therefore, the embodiments shall be considered as exemplary and unrestricted in any way. The scope of the utility model is defined by the appended claims rather than the above description. Hence, all changes intended to come within the meaning and range of equivalent elements of the claims shall be included within the utility model. Any marks on drawings to the Claims shall not be construed as limiting the Claims involved.

Furthermore, it shall be understood that although the Specification is described in terms of embodiments, not every embodiment includes only one independent technical scheme. The description style in the Specification is for clarity only. Those skilled in the art shall take the Specification as a whole. The technical schemes in various embodiments may also be combined as appropriate to form other embodiments that can be understood by those skilled in the art.

1. A headphone with motion sensors, comprising a left channel earphone, a right channel earphone and a main electronic circuit, wherein the motion sensors are provided in the main electronic circuit, the left channel earphone and the right channel earphone respectively, and are all linked to a microprocessor.

2. A headphone with motion sensors according to claim 1, wherein the main electronic circuit is further provided with a microprocessor, a D/A conversion circuit, an amplifier and a Bluetooth module; a speaker is disposed in the left channel earphone- and the right channel earphone respectively, and is connected to the amplifier that is connected with the D/A conversion circuit; the D/A conversion circuit is linked to the microprocessor.

3. A headphone with motion sensors according to claim 1, wherein the main electronic circuit is disposed within the headphone beam.

4. A headphone with motion sensors according to claim 1, wherein the main electronic circuit is further provided with a vibrator that is connected to the microprocessor.

5. A headphone with motion sensors according to claim 1, wherein the said motion sensors are all connected to the microprocessor, and the headset design determines the number and position of the motion sensors.
